(19) United States
(12) Patent Application Publication
(10) Pub. No.: US 2003/0135185 A1
(43) Pub. Date: Jul. 17, 2003

(54) Adhesive-less absorbent garment

(76) Inventor: David George Crowther, Suwanee, GA (US)

Correspondence Address: Christopher C. Campbell, Esq., HUNTON & WILLIAMS, 1900 K. Street, N.W., Washington, DC, 20006-1109, US

(21) Appl. No.: 10/052,604

(22) Filed: Jan. 17, 2002

(51) Int. Cl. A61F 13/15

(52) U.S. Cl. 604/385.01; 604/386

(57) ABSTRACT

An absorbent garment in which the various component parts are held in position with respect to each other by one or more adhesive-less bonds is disclosed. A method and apparatus for assembling the garment are also disclosed.

### BACKGROUND OF THE INVENTION

1. Field of the Invention

The invention relates generally to absorbent garments. More particularly this invention relates to an absorbent garment in which the various component parts are held in position with respect to each other by one or more adhesive-less bonds and to a method for assembling such a garment without application of adhesives in the assembly process.

2. Background of the Invention

Disposable absorbent garments such as infant diapers or training pants, adult incontinence products, and other such products are well-known in the art. Typically, the chassis of such garments comprises a liquid-permeable body-contacting liner sheet (or “top sheet”), a liquid-impermeable backing sheet (or “back sheet”) (collectively the “sheets”), and a moisture-absorbent core fiber (or “absorbent core”) that usually is made of a non-woven mat of randomly arranged fiber and is generally disposed between the top sheet and the back sheet. These garments typically further comprise various elastic components and fastening systems to facilitate use of the garments, and they often comprise additional sheets.

Disposable absorbent articles generally are assembled on an automated production line by separately supplying the individual components of the absorbent article to the production line at predetermined locations along the machine direction, and layering the individual components to form an integrated absorbent article. Various methods are available for bringing these individual components together so that the components in the integrated product are in a desired relation with respect to each other. In bringing these individual components together, adhesives typically are used to secure the placement of a component in the desired position with respect to the position of other components.

In a typical commercial disposable absorbent article such as a diaper, a dozen or more distinguishable applications of adhesive may be used during the process of assembling the garment. These include substantially entirely coating the back sheet and one or more of the other sheets, coating a major portion of at least one surface of the absorbent core, coating a portion of other sheets, and multiple applications of adhesive to secure various elastic components and fastener system components. Typically adhesive is liberally applied in an overspray process.

The absorbent garment materials that are “oversprayed,” such as portions of the top sheet, the bottom sheet, and the absorbent core, become rigid upon hardening of the “oversprayed” adhesive making the garment less comfortable for its wearer. This has been referred to as the “plywood effect.” The “overspray” also coats parts of the assembly machinery that must then be periodically cleaned of the adhesive. Further specialized components such as the elastic elements may not be uniformly coated with the adhesive due to the nature of the spraying operation, and therefore the elastic elements may not bond to the garment materials as well as if they had been more uniformly and completely coated with the adhesive.

Adhesive applied onto the fabric may also reduce the fluid absorbing capacity of the fabric and possibly result in the leakage of bodily fluids from the absorbent garment. Additionally, the adhesive stiffened fabric may be slightly abrasive against the skin, and in some cases may irritate sensitive skin.

The production of a representative four stage commercially available diaper requires approximately twelve distinctive applications of adhesive using approximately 1.75 grams of adhesive per diaper. Thus adhesives are a significant materials cost in the production of such diapers. In addition to direct materials costs, adhesives, particularly the hot melt adhesives that are typically used, may contribute to production costs due to fouling of dispensing systems and contamination of equipment or materials. These costs may be due to down time to repair or clean equipment or both or due to the production of defective articles such as articles where components are not secured due to lack of adhesive or garments that are contaminated by extraneous adhesive. The typically used hot melt adhesives may also deform or even destroy the relatively thin temperature sensitive fabric if too much hot adhesive is applied.

Due to the need to adequately secure the various components and the challenges in applying adhesives, more adhesive than the minimum amount to form a bond is frequently used to compensate for such inefficiency. More adhesive is also usually used to maintain a desirable production rate for garments in the assembly process. This further increases material costs.

Further, many of the positions of use of adhesive are internal in the finished absorbent garment, thus many of the steps of applying adhesive must be done sequentially. Additionally it is frequently preferable to feed materials into an assembly line in a manner and at a physical position that avoids unwanted contact with adhesives to minimize contamination.

The foregoing description of the various products, methods, and apparatus, and their attendant disadvantages is in no way intended to limit the scope of the present invention, or to imply that the present invention does not include some or all of the various elements of the products, methods, and apparatus in one form or another. Indeed, various embodiments of the invention may be capable of overcoming some of the disadvantages, while still retaining some or all of the various elements of the products, methods, and apparatus in one form or another.

### SUMMARY OF INVENTION

It is therefore a feature of various embodiments of the invention to address the aforementioned needs by providing an absorbent garment and a method for making an absorbent garment using adhesive-less bonding for assembling the absorbent garment and affixing the component parts in a position with respect to each other. In a manufacturing setting, the method provides for making absorbent garments on a production line free of adhesive deposition devices and the dispensing of liquid glues.

In accordance with these and other features, there is provided an absorbent garment having a longitudinal and a lateral dimension comprising a top sheet, a back sheet and absorbent core disposed between the top sheet and back sheet whereby the top sheet, back sheet and absorbent core are held in a position with respect to each other by at least one adhesive-less bond. In one exemplary embodiment at least a portion of the adhesive-less bond is an ultrasonic bond. In another exemplary embodiment at least a portion of the adhesive-less bond is a thermal bond. The top sheet and back sheet form a first waist region, a second waist region longitudinally opposite the first waist region and a crotch there between. In some embodiments the absorbent core has a perimeter edge and an adhesive-less bond between the top sheet and back sheet is adjacent a portion of the perimeter edge.

The absorbent garment may have additional layers attached to the garment by adhesive-less bonds comprising at least one of an outer top sheet panel, back sheet panel, liner, transfer layer, chassis, laminate and graphics panel. The absorbent garment may also have tabs and elastic components attached which are preferably attached to the absorbent garment by adhesive-less bonds. In other embodiments the tabs, elastic or both may be supplied as a laminate that is attached to the absorbent garment by an adhesive-less bond.

In accordance with another embodiment of the invention, an apparatus for making an absorbent garment is provided. The apparatus for making an absorbent garment, according to one embodiment of the invention comprises: a top sheet supply mechanism for supplying a top sheet, a back sheet supply mechanism for supplying a back sheet; an absorbent core supply mechanism for supplying an absorbent core; a forming station which receives the top sheet from the top sheet supply mechanism, the back sheet from the back sheet supply mechanism and the absorbent core from the absorbent core supply mechanism and disposes the absorbent core between the top sheet and back sheet and; at least one adhesive-less bonding station that creates at least one adhesive-less bond to hold the top sheet, the back sheet and the absorbent core in a position with respect to each other thereby forming an absorbent garment.

The apparatus for making an absorbent garment may further comprise a fastener system supply mechanism for supplying a fastener system and a fastener system attachment station for attaching the fastener system to the absorbent garment with at least one adhesive-less bond.

The apparatus for making an absorbent garment may also comprise an elastic strand supply mechanism and an elastic strand attachment station. In one exemplary embodiment the elastic strand supply mechanism supplies at least one elastic strand and the elastic strand attachment station receives at least one elastic strand and attaches it to the absorbent garment by an adhesive-less bond. Alternatively the elastic strand supply mechanism may supply at least one elastic strand disposed in a laminate and the elastic strand attachment station attaches the laminate to the absorbent garment by at least one adhesive-less bond. In some embodiments the elastic strand attachment station may attach a plurality laminates having at least one elastic strand disposed in each laminate.

The invention further includes a method for manufacturing an absorbent garment comprising providing a top sheet, a back sheet and an absorbent core, disposing the absorbent core between the top sheet and the back sheet and fixing the top sheet, back sheet and absorbent core in a position with respect to each other with at least one adhesive-less bond to form an absorbent garment. The method may include attaching one or more additional layers comprising at least one of an outer top sheet panel, chassis, back sheet panel, liner, transfer layer, laminate and graphics panel to the absorbent garment by at least one adhesive-less bond.

The method for manufacturing an absorbent garment may further comprise attaching at least one fastener system to the absorbent garment with at least one adhesive-less bond. In another exemplary embodiment the method for manufacturing an absorbent garment further comprises attaching at least one elastic strand to the absorbent garment.

### BRIEF DESCRIPTION OF THE DRAWINGS

The invention may be understood more readily by reviewing the drawings, in which:

FIG. 1 shows a partially cut away view of an exemplary embodiment of an absorbent garment of the invention;

FIG. 2 shows a view of the top sheet of an exemplary embodiment of an absorbent garment of the invention;

FIG. 3 shows a view of the back sheet of an exemplary embodiment of an absorbent garment of the invention; and

FIG. 4 is a schematic of an apparatus for making an absorbent garment according to an exemplary embodiment of the invention.

### DETAILED DESCRIPTION OF PREFERRED EMBODIMENTS

One advantage of one embodiment of the invention is the production of an absorbent garment with improved softness and comfort to the wearer by avoiding overspray or other extensive coating of adhesives. The sheets and absorbent core retain their original fabric softness and pliability, rather than becoming partially rigid through stiffening of the material upon hardening of the adhesive.

Another advantage of the invention is a reduction of material cost in the production of an absorbent garment, by in some embodiments, eliminating the use of adhesives and in other embodiments limiting the use of adhesives to preparation of preformed components.

A further advantage of the invention is an improvement in manufacturing efficiency in production of an absorbent garment by eliminating adhesive dispensing devices from the absorbent garment assembly production line. Adhesive dispensing devices are prone to fouling which create maintenance issues and contamination of equipment. Further problems associated with improper function of adhesive dispensing devices contribute significantly to lost product due to production defects such as contamination from extraneously applied adhesive or garments with components not properly secured due interruption in the dispensing of adhesive due to clogging of the dispensing equipment.

Another advantage of the invention is that the method of the invention for making an absorbent garment using adhesive-less bonding may be implemented with a system of feeding materials into an assembly line of the type commonly used in commercial production of absorbent garments. Because there is no requirement that internal layers be secured before outer layers are added to the absorbent garment, alternative assembly procedures also may be used. For example, it is not necessary to secure the absorbent core into position with at least one adjacent layer prior to positioning other layers. When applying adhesives the typical practice is to fix two layers which must be held in a position with respect to each other by applying adhesive to one or both, selecting the position and contacting the adhesive coated layer(s) before positioning other layers over either of the first two layers. In the method of the invention multiple layers may be assembled and bonded with a single bonding event.

A further advantage is that adhesive-less bonds may be used to secure a variety of optional components such as graphics bearing layers, for example, either independently or in combination with other layers in a single bonding event.

Another advantage is that adhesive-less bonds may positively contribute to the aesthetics of the absorbent garment by being formed in decorative patterns, providing texture that improves the hand (e.g. “feel”) of the garment fabric or both, for example.

The invention provides an absorbent garment having a longitudinal and a lateral dimension comprising a top sheet and a back sheet, whereby the top sheet and the back sheet form a first waist region, a second waist region longitudinally opposite the first waist region with a crotch there between. An absorbent core may be disposed between the top sheet and back sheet whereby the top sheet and back sheet and absorbent core are held in a position with respect to each other by at least one adhesive-less bond. In one exemplary embodiment the at least one adhesive-less bond is an ultrasonic bond. In another exemplary embodiment the at least one adhesive-less bond is a thermal bond.

The absorbent core preferably has a perimeter edge and the adhesive-less bond between the top sheet and the back sheet may be positioned adjacent a portion of the perimeter edge, thereby securing the position of the absorbent core. In some embodiments the back sheet is a film comprising at least one of polyethylene, polypropylene, polyester, nylon, and polyvinyl chloride. In other embodiments, the top sheet may be a non-woven spunbonded or carded web of at least one of polypropylene, polyethylene, nylon and polyester. The absorbent garment may further comprise at least one layer, the layer comprising at least one of an outer top sheet panel, chassis, back sheet panel, liner, transfer layer, laminate, or graphics panel, attached to the absorbent garment by an adhesive-less bond.

The absorbent garment may further comprise a pair of tabs extending laterally away from opposite lateral sides of the first waist region. At least a portion of each tab includes a fastening element. One or more target devices may be attached to the garment in the second waist region. The fastening elements and the target devices are capable of attaching to one another. The target devices preferably are located so that the first and second waist region of the garment may be joined to secure the garment on the intended wearer. The fastening element(s) and target device(s) are attached to the absorbent garment by an adhesive-less bond. In one exemplary embodiment the tabs are individual components attached to the absorbent garments by adhesive-less bonds.

The garment may further comprise at least one elastic strand wherein the elastic strand is attached by an adhesive-less bond. In some embodiments the elastic strand is precoated with a polymer, the polymer upon activation forming a bond between the elastic strand and the absorbent garment. In another embodiment, the elastic strand may be extruded onto the absorbent garment and adhered to the absorbent garment. In another exemplary embodiment the elastic strand may be disposed in a laminate comprising a first substrate and a second substrate. The laminate is attached to the absorbent garment by an adhesive-less bond, but the elastic strands may be attached to the first and/or second substrate in the laminate by an adhesive.

The invention also includes an apparatus for making an absorbent garment. The apparatus comprises a top sheet supply mechanism for supplying a top sheet; a back sheet supply mechanism for supplying a back sheet and an absorbent core supply mechanism for supplying an absorbent core. The apparatus also comprises a forming station, which receives the top sheet from the top sheet supply mechanism, the back sheet from the back sheet supply mechanism and the absorbent core from the absorbent core supply mechanism and which disposes the absorbent core between the top sheet and the back sheet. The apparatus also includes at least one adhesive-less bonding station that creates at least one adhesive-less bond to hold the top sheet, the back sheet and the absorbent core in a position with respect to each other. In one exemplary embodiment a portion of the at least one adhesive-less bond is an ultrasonic bond. In another embodiment a portion of the at least one adhesive-less bond is a thermal bond. The apparatus in these embodiments therefore would include, as the at least one adhesive-less bonding station, an ultrasonic and/or thermal bonding apparatus.

The apparatus for making the absorbent garment using adhesive-less bonding may further comprise a fastener system supply mechanism for supplying a fastener system and a fastener system attachment station for attaching the fastener system to the absorbent garment with at least one adhesive-less bond. The fastener supply system may supply at least one fastening element and at least one target device. The fastener system attachment station receives the at least one of the fastening element and the target device and attaches the at least one fastening element to a tab that is an extension portion of at least one of the top sheets and back sheets and attaches the at least one target device to a second portion of at least one of the top sheets and back sheets. The fastener attachment station may have a fastener element attachment portion and a target device attachment portion.

In one exemplary embodiment the fastener system supplies a fastening element and a target device wherein at least one of the at least one fastening element and the at least one target device is attached to at least one separate component tab by an adhesive-less bond. The fastener system attachment station attaches the at least one separate component tab to the absorbent garment by an adhesive-less bond.

The apparatus for making the absorbent garment may further comprise an elastic strand supply mechanism and an elastic strand attachment station. The elastic strand supply mechanism supplies at least one elastic strand. The elastic strand attachment station receives at least one elastic strand and attaches it to the absorbent garment by an adhesive-less bond.

In one embodiment the apparatus for making an absorbent garment may include an elastic strand supply mechanism and an elastic strand attachment station wherein the elastic strand supply mechanism supplies an elastic strand precoated with a polymer. Upon activation the polymer forms a bond between the elastic strand and the absorbent garment.

In another embodiment the apparatus for making an absorbent garment may include an elastic strand supply mechanism and an elastic strand attachment station wherein the elastic strand is extruded from the elastic supply mechanism onto a portion of the absorbent garment at a portion of the elastic strand attachment station.

In another embodiment the elastic strand supply mechanism supplies at least one elastic strand disposed in an elastic strand laminate. The laminate comprises a first substrate and a second substrate with the at least one elastic strand disposed between the first and second substrate. The laminate is supplied to the elastic strand attachment station, and the elastic strand attachment station attaches the laminate to the absorbent garment with at least one adhesive-less bond. In some embodiments a plurality of elastic strand laminates may be attached to the absorbent garment by at least one adhesive-less bond.

The invention further provides a method for manufacturing an absorbent garment. The method for manufacturing an absorbent garment includes providing a top sheet, a back sheet and an absorbent core. The absorbent core is disposed between the top sheet and the back sheet. The top sheet, back sheet and absorbent core are fixed in a position with respect to each other with at least one adhesive-less bond.

In one exemplary embodiment a portion of the adhesive-less bond is an ultrasonic bond. In another embodiment a portion of the adhesive-less bond is a thermal bond.

The method for manufacturing an absorbent garment may further comprise attaching a layer comprising at least one of an outer top sheet panel, chassis, back sheet panel, liner, transfer layer, laminate and graphics panel to the absorbent garment by an adhesive-less bond.

The method of manufacture may further comprise attaching a fastener system to the absorbent garment with at least one adhesive-less bond. The fastener system may include at least one fastening element and at least one target device and at least one of the fastening element and target device are attached to a tab that is an extension of at least one of the top sheet and back sheet. In another embodiment at least one of the fastening element and target device is attached to at least one separate component tab. The separate component tab is attached to the absorbent garment by an adhesive-less bond.

The method for manufacturing the absorbent garment may further comprise attaching at least one elastic strand to the absorbent garment by an adhesive-less bond. In one embodiment the elastic strand may be extruded onto a portion of the absorbent garment. In another embodiment the elastic strand may be precoated with a polymer that upon activation forms a bond between the elastic strand and absorbent garment.

In other embodiment the at least one elastic strand is disposed in a laminate comprising a first substrate and a second substrate. The elastic strand is disposed between the first and second substrates. The laminate is attached to the absorbent garment by an adhesive-less bond. In some embodiments a plurality of laminates may be attached to the absorbent garment by one or more an adhesive-less bond.

The invention provides a core assembly for an absorbent garment comprising a top sheet, a back sheet and an absorbent core. The core is disposed between the top sheet and back sheet and the core has a perimeter edge. The top sheet and back sheet are bonded in an adhesive-less bond adjacent a portion of the perimeter edge thereby substantially holding the absorbent core in a selected position with respect to the top sheet and the back sheet. In one embodiment a portion of the at least one adhesive-less bond is an ultrasonic bond. In some embodiments a portion of the adhesive-less bond is a thermal bond.

As used herein, the term “absorbent garment” refers to garments that absorb and contain exudates, and more specifically, refers to garments, which are placed against or in proximity to the body of the wearer to absorb and contain the various exudates discharged from the body. A nonexhaustive list of examples of absorbent garments includes diapers, diaper covers, disposable diapers, training pants, feminine hygiene products and adult incontinence products. The term “disposable absorbent garment” refers to absorbent garments that are intended to be discarded or partially discarded after a single use (i.e., they are not intended to be laundered or otherwise restored or reused). The term “unitary disposable absorbent garment” refers to a disposable absorbent garment that is essentially a single structure (i.e., it does not require separate manipulative parts such as a diaper cover and insert). As used herein, the term “diaper” refers to an absorbent garment generally worn by infants and incontinent persons about the lower torso.

The claims are intended to cover all of the foregoing classes of absorbent garments, without limitation, whether disposable, unitary or otherwise. These classifications are used interchangeably throughout the specification, but are not intended to limit the claimed invention. The invention will be understood to encompass, without limitation, all classes of absorbent garments, including those described above. Preferably, the absorbent core is thin in order to improve the comfort and appearance of a garment.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

As used herein “adhesive-less” means that one component part is affixed (directly or indirectly) to another component part or parts without use of a discrete intermediary composition positioned between the first component and the one or more other components that joins at least a portion of the surface of the components in a substantially fixed position with respect to each other (e.g. no adhesive is applied during assembly of the absorbent garment). As used herein an adhesive means a discrete substance or composition that joins at least a portion of the surfaces of two or more components in a fixed position with respect to each other. In preferred embodiments, no adhesive is used in assembly of the absorbent garment or in the formation of component parts. In other embodiments preformed laminates may be used that, as supplied, have been formed using adhesives, but no adhesive is applied during the assembly of the laminate with other components to form the garment, e.g. the laminate is affixed directly to one or more other component parts of the garment without use of an intermediate bonding material positioned between the laminate and one or more other components. In other embodiments certain components may be made of or coated with materials that may be activated by heat, pressure, UV light or the like to adhere to a substrate. In these cases the material forming the bond is an integral part of at least one of the components.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Absorbent garments and diapers may have a number of different constructions. In each of these constructions it is generally the case that an absorbent core is disposed between a liquid pervious body-facing top sheet, and a liquid impervious, exterior facing back sheet. In some cases, one or both the top sheet and the back sheet may be shaped to form a pant-like garment. In other cases, the top sheet, back sheet and absorbent core may be formed using a discreet assembly that is placed on a main chassis and the chassis is made to form a pant-like garment. In the case of diapers, a caregiver usually wraps the diaper around the wearer's waist and joins the side seams manually by attaching one or more adhesive or mechanical tabs, thereby making the pant-like structure. In the case of training pant-type garments and most adult incontinent products, the garment is provided fully formed with factory made side seams and the garment is donned by pulling it up the wearer's leg. For clarity, the present invention is described herein, with reference to the diaper type garment; although the invention may be used with other constructions having elastics incorporated therein including for example, the training pant-type garments, adult and incontinent products or feminine hygiene products.

The invention now will be described with reference to the attached drawings illustrating preferred embodiments of the invention. For clarity, features that appear in more than one Figure have the same reference number in each Figure.

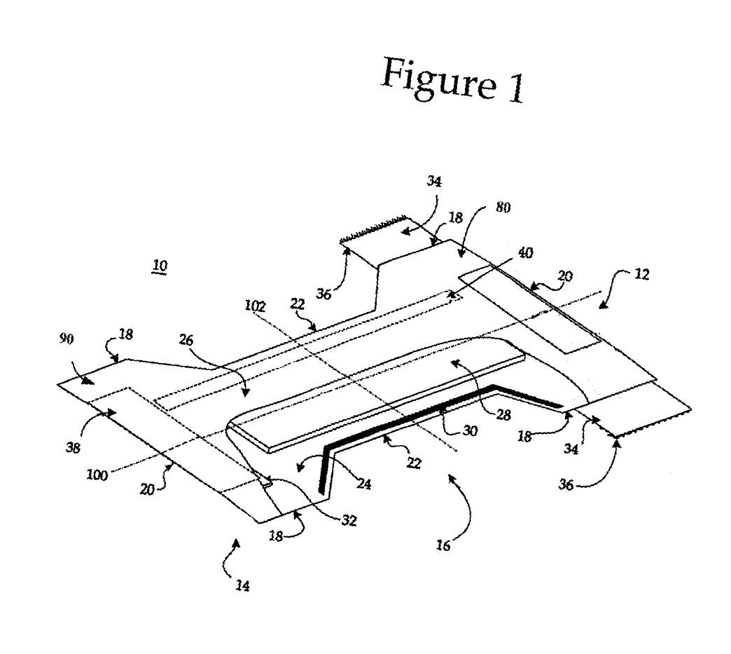

FIG. 1 is a partially cut away depiction of an exemplary embodiment of an absorbent garment 10 (preferably a disposable absorbent garment) of the present invention. The embodiment shown in FIG. 1 is an infant's diaper, however, this depiction is not intended to limit the invention, and those skilled in the art appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their extended condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 form the perimeter of a pair of leg openings, when the garment 10 is placed on a subject wearer.

The garment 10 preferably comprises a top sheet 24, and a back sheet 26, which may be substantially coterminous with the top sheet 24. When the garment 10 is being worn, the top sheet 24 faces the wearer's body, and the back sheet 26 faces away from the wearer. An absorbent core 28 preferably is disposed between at least a portion of the top sheet 24 the back sheet 26.

An embodiment of the present invention may further comprise various additional features. One or more pairs of elastic gathers 30 may extend adjacent the crotch edges 22 or a portion thereof. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of elastic waist foam 32 or other elastically extensible material, which help contract the garment around the wearer's waist, providing improved fit and leakage prevention.

The absorbent garment 10 also preferably includes fastening elements to enable attachment of the first waist region 12 to second waist region 14. Fastening elements preferably include a pair of tabs 34 that extend laterally away from opposite lateral edges 18 of the first waist region 12 of the garment 10. The tabs 34 maybe components attached to one or both of the back sheet 26 and top sheet 24, alternatively, the tabs 34 may be formed as a part of another component such as top sheet 24 or back sheet 26 or a combination thereof. The tabs 34 may comprise an elastically extensible material (not shown), and may be designed to stretch around a wearer's waist to provide improved fit, comfort, and leakage protection. Such elasticized tabs 34 may be used in conjunction with, or in lieu of, waist foam 32, or other elastically extensible materials 32.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14.

Although not shown in the drawings, the absorbent garment 10 may also include grips (also referred to as finger lifts) attached along one of its edges proximal to each tab 34 to enable a caregiver to pull the grips, and not on the ends of the tabs 34, around the wearer and over the target devices 38 to thereby secure the fasteners 36 to the one or more target devices 38.

Optionally, the absorbent garment 10 may include graphic panels (not shown). Such graphic panels may be decorative and improve aesthetics or may facilitate use of the garment such as indicating front and back orientation or providing wetness indicator. The graphics panels typically are disposed on the exterior of the garment (i.e., on back sheet 26, or outer layer), or they may be present interior to the outer most layer if the outer most layer is transparent or translucent.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate component parts, and by fixing the relative positions of various parts by capturing parts between other parts. As noted above, component parts are held in fixed positions by the attachment of one component to one or more component parts by formation of an adhesive-less bond or are held in position by an integral portion of one component interacting with a portion of one or more other components.

In preferred embodiments at least a portion of the bonds are formed using ultrasonic energy. Ultrasonic bonding and embossing techniques are disclosed in, for example, U.S. Pat. No. 3,562,041 (ultrasonic joining of materials according to a pattern), U.S. Pat. No. 3,733,238 (vibration welding of sheet materials), U.S. Pat. No. 4,668,316 (welding thin thermoplastics film by ultrasonic energy), U.S. Pat. No. 5,096,532 (ultrasonic rotary horn), U.S. Pat. No. 5,110,403 (high efficiency ultrasonic rotary horn), U.S. Pat. No. 5,846,377 (welding thermoplastic work pieces using ultrasonic energy), U.S. Pat. No. 6,036,796 (ultrasonic welding apparatus and method), U.S. Pat. No. 6,165,298 (patterned anvil roll to effect bonding, cutting, embossing, perforating) and U.S. Pat. No. 6,277,224 (ultrasonic perforator), the disclosures of each of which is incorporated by reference herein in their entirety to the extent that they are consistent with this disclosure.

Alternatively, thermal bonding devices such as heated rolls may be used to bond components in some embodiments. The use of heated rolls to thermally emboss a web of material have been disclosed in U.S. Pat. Nos. 5,229,186 and 6,041,701 the disclosures of each of which are incorporated by reference herein in their entirety to the extent that they are consistent with this disclosure.

In other embodiments, pressure or pressure in combination with heat may be used to bond components. Plural laminae may be bonded by this method if at least one laminae comprises a thermoplastic material. The method comprises forwarding the laminae through a nip between a patterned nip defining member and a nip defining anvil member, and pressure biasing the nip defining members towards each other with a predetermined pattern-element-psi loading and rotating the nip defining members to provide a predetermined surface velocity differential therebetween. The method may optionally further include heating the nip defining members. The use of pressure and pressure in combination with heating to bond layers of material, at least one of which is a thermoplastic material, have been disclosed in U.S. Pat. Nos. 4,854,984 and 4,919,738, the disclosures of which are incorporated by reference herein in their entirety.

In other embodiments an integral part of the composition of a component may be amenable to attachment to another component upon activation. For example a component may have a polymeric portion that under specified conditions (e.g. activation) may form a bond. Activation may include the application of pressure, heat or UV radiation, for example.

The top sheet 24 and back sheet 26 may be constructed from a wide variety of materials known in the art. Due to the wide variety of backing and liner sheet construction and materials currently available, the invention is not intended to be limited to any specific materials or constructions of these components. The top sheet 24 and back sheet can be shaped and sized according to the requirements of each of the various types of absorbent garment, or to accommodate various user sizes. In an embodiment of the invention in which the garment 10 is a diaper or an adult incontinence brief, the combination of top sheet 24 and back sheet 26, may have an hourglass shape, as seen in FIG. 1, or may have a rectangular, trapezoidal, “T” shape, or other shape.

The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 26 may be pigmented with, for example, titanium dioxide, to provide the garment 10 with a pleasing color or to render the back sheet 26 opaque enough that exudates being contained by the garment 10 are not visible from outside the garment. In addition, the back sheet 26 may be formed in such a manner that it is opaque, for example, by using various inert components in the polymeric film and then biaxially stretching the film. Other back sheet materials will be readily apparent to those skilled in the art. The back sheet 26 preferably has sufficient liquid imperviousness to prevent any leakage of fluids. The required level of liquid imperviousness may vary between different locations on the garment 10.

The back sheet 26 may further comprise separate regions having different properties. In a preferred embodiment, portions of the back sheet 26 are air-permeable to improve the breathability, and therefore comfort, of the garment 10. The different regions may be formed by making the back sheet 26 a composite of different sheet materials, chemical treatment, heat treatment, or other processes or methods known in the art. Some regions of the back sheet 26 may be fluid pervious. In one embodiment of the invention, the back sheet 26 is fluid impervious in the crotch 16, but is fluid pervious in portions of the first and second waist regions 12, 14. The back sheet 26 may also be made from a laminate of overlaid sheets of material. In embodiments having a back sheet 26 made from a laminate of over laid sheet materials it is preferable that the sheets are held in position with respect to each other by one or more adhesive-less bond.

The back sheet 26 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet 26 may comprise three panels wherein a central poly back sheet panel is positioned closest to absorbent core 28 while outboard non-woven breathable side back sheet panels are attached to the side edges of the central poly back sheet panel. In embodiments having a back sheet 26 comprised of three panels it is preferable that the panels be fixed in their relative positions to each other by adhesive-less bonds. Alternatively, the back sheet 26 may be formed from microporous poly coverstock for added breathability.

The top sheet 24 may be made of any suitable relatively liquid-pervious material currently known in the art or later discovered that permits passage of a liquid there through. In some embodiments it is preferable that the top sheet 24 be made of materials that are amenable to ultrasonic or thermal bonding. Examples of suitable top sheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 28. The top sheet 24 preferably comprises a single-ply nonwoven material that may be made of carded fibers, preferably or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 24, and other top sheet materials will be readily apparent to those skilled in the art.

The top sheet 24 may further comprise several regions having different properties. In one embodiment of the present invention, the laterally distal portions of the top sheet 24, especially those used to make the outer top sheet panels preferably are substantially fluid impervious and hydrophobic, while the remainder of the top sheet 24 (e.g., central top sheet panel) is hydrophilic and fluid pervious. Different top sheet properties, such as fluid perviousness and hydrophobicity, may be imparted upon the top sheet 24 by treating the top sheet 24 with surfactants, or other chemicals, using a composite of different materials, or by other means. The top sheet 24 may also be made from a laminate of overlaid sheets of material. The top sheet 24 also may be treated in specific areas like the crotch region, with skin wellness ingredients such as aloe, vitamin E, and the like.

The top sheet 24 may be formed of three separate portions or panels. In embodiments in which the top sheet 24 is formed from three separate portions or panels, it is preferable that the panels or positions be fixed in their relative positions to each other by one or more adhesive-less bonds. Those skilled in the art will recognize, however, that top sheet 24 need not be made of three separate panels, and that it may be comprised of one unitary item. A first top sheet panel (not shown) may comprise a central top sheet panel formed from preferably a liquid-pervious material that is either hydrophobic or hydrophilic. The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, preferably thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet panel preferably extends from substantially the second waist region 14 to the first waist region 12, or a portion thereof.

A second and third top sheet panels (e.g., outer top sheet panels, not shown in FIG. 1), in this alternative embodiment may be positioned laterally outside of the central top sheet panel. The outer top sheet panels preferably are substantially liquid-pervious, preferably at least in the crotch area. In some embodiments selected portions of the outer top sheet panels may be liquid-impervious and hydrophobic. The outer edges of the outer top sheet panels may substantially follow the corresponding outer perimeter of the back sheet 26. The material for the outer top sheet portions or panels is preferably polypropylene and can be woven, nonwoven, spunbonded, carded or the like, depending on the application. In some embodiments it is preferable that top sheet 24 materials including top sheet panels are materials that are amendable to ultrasonic or thermal bonding.

As noted elsewhere herein, the top sheet 24 and back sheet 26 may be substantially coterminous, or they may have different shapes and sizes. The particular design of the top sheet 24 and back sheet 26 may be dictated by manufacturing considerations, cost considerations, and performance considerations. Preferably, the top sheet 24 is large enough to completely cover the absorbent core 28, and the back sheet 26 is large enough to prevent leakage from the garment 10. The design of top sheet 24 and back sheet 26 is known in the art, and a skilled artisan will be able to produce an appropriate top sheet 24 and an appropriate back sheet 26 without undue experimentation.

The top sheet 24 and the back sheet 26 preferably are associated with one another using one or more adhesive-less bonds. For example, an integral portion of the top sheet 24, back sheet 26 or both may be thermally or ultrasonically bonded. The particular joining method may be dictated by the types of materials selected for the top sheet 24 and back sheet 26.

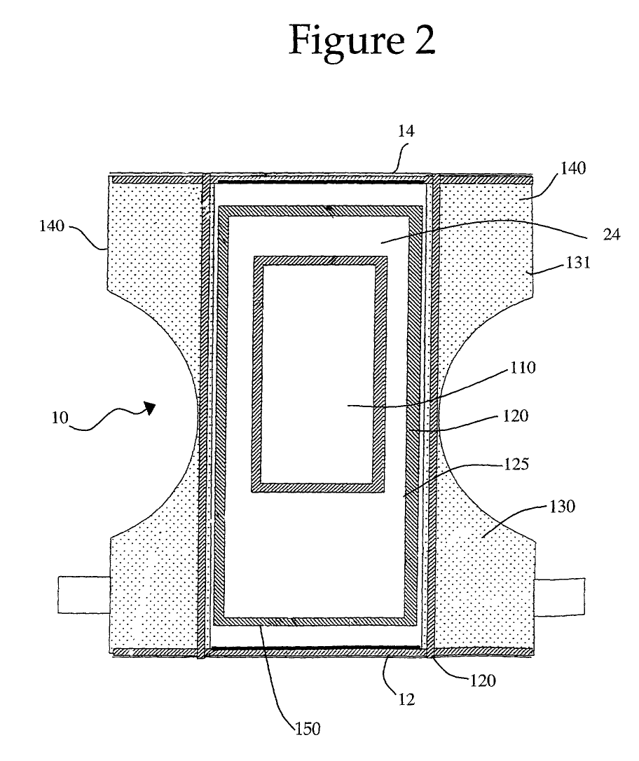

A single adhesive-less bond or multiple adhesive-less bonds may be used. In one exemplary embodiment shown in FIG. 2 ultrasonic bonding is used to affix the components in the desired position with respect to each other. The dissipation of ultrasonic energy in a predetermined pattern causes the material of the top sheet 24, back sheet 26 and other layers in the path of the predetermined pattern to become pliable and to achieve sufficient tackiness that they adhere to each other. In some embodiments, especially in the embodiments having thermoplastic materials, the energy imparted may be sufficient such that the layers become partially fluid and melt together forming a region in which no individual layer of material may be distinguished. The pre-determined pattern may be a fused region (e.g. seam weld) or a group of fused points (e.g. pin bonds). In the embodiment shown in FIG. 2 a combination of seam welds 120 and pin bonds 130 are used. The seam weld 120 may be a simple linear bond as shown in FIG. 2 or follow a curved pattern that yields an embossed appearance. The embossed pattern may add desirable aesthetic features such as improved appearance or improved hand (e.g. feel of the material to touch) or both. In some embodiments the positioning and/or width of seam welds may contribute re-enforcement properties in areas of higher in use stress.

Pin bonds 130 can be made by using pins when imparting the ultrasonic energy to the garment material. Typically a plurality of pins are used with the end of each pin that touches the fabric having a small surface area. Bonding occurs at the position touched by each pin. Typically when using pins the amount of material bonded together when compared to the total surface area of material contact is small. This method may be effective for holding materials in position with respect to each other while maintaining fabric softness. Those skilled in the art are capable of designing without undue experimentation a suitable method and apparatus to use pin bond 130 to attach the respective components to one another, using the guidelines provided herein.

FIG. 2 also shows a transfer layer 110 bonded to a top sheet 24. The top sheet 24 is bonded to a back sheet 26 underlying the top sheet 24 and as shown is coterminous with the top sheet 24 and not visible in FIG. 2. Seam welds 120 also can be provided along the first waist region 12 and second waist region 14, around the perimeter of the transfer sheet 110, and parallel to the longitudinal axis 100. Additionally, a core assembly seam weld 150 between the top sheet 24 and back sheet 26 around at least a portion of the perimeter 125 of the absorbent core 28 is provided. In one embodiment core assembly seam weld 150 bonds top sheet 24 and back sheet 26 materials together forming a pocket that holds the absorbent core 28 in position. The seam welds 120 may be varied in width and length and may follow the linear pattern shown or a curved pattern. Factors that may be considered in determining width and pattern of the seam weld 120 are number of layers to be bonded, materials to be bonded, stress applied to bond when the garment is used in use and appearance, for example. Skilled artisans are capable of determining a suitable width and pattern for seam welds 120, using guidelines provided herein.

The seam welds 120 may be formed sequentially e.g. transfer layer 110 may be bonded to the top sheet 24 prior to bonding the top sheet 24 to the back sheet 26, for example. Alternatively materials may be assembled and one or more seam welds executed at a single bonding station with two or more layers bonded in a single bonding event or the bonding of multiple positions being executed substantially simultaneously or both.

In the exemplary embodiment shown in FIG. 2, pin bonding 130 is used to bond the ear portions 140 of the top sheet 24 and back sheet 26 together. In this embodiment points 131 indicate the positions of attachment between the top sheet 24 and the back sheet 26. The total amount of surface area of the top sheet 24 and back sheet 26 bonded is small by comparison to the total surface area in contact. The distribution of the points 131 over the entire surface area serves to secure the top sheet 24 in position with respect to back sheet 26, while the limited area engaged in forming the bonds provides for retention of the fabric softness.

Pattern and position of bonding may be selected with considerable selectively, specification and precision. Thus, the amount and position of bonding can be optimized for securing components while minimizing area engaged in bonding and hence minimizing stiffness typically associated with bonding.

Again referring to FIG. 1, garment 10 may include outer top sheet portions or panels that may extend upwardly to form waste containment flaps 40. The waste containment flaps 40 preferably are formed of the same material as the outer top sheet portions or panels, as in the embodiment shown. The waste containment flaps 40 may be treated with a suitable surfactant to modify their hydrophobicity/hydrophilicity as desired, and they may be treated with skin wellness ingredients to reduce skin irritation. Alternatively, the waste containment flaps 40 may be formed as separate elements and then attached to the body side liner, top sheet 24, chassis, back sheet 26, and the like.

The waste containment flaps 40 preferably include a portion that folds over onto itself to form a small enclosure. At least one, and depending on the size of the enclosure sometimes more than one, elastic member may be secured in the enclosure in a stretched condition. When the flap elastic attempts to assume the relaxed, unstretched condition, the waste containment flaps 40 rise above the surface of the central top sheet portion or panel.

As mentioned above, the absorbent garment preferably is provided with leg elastics 30 extending through crotch region 16 or a portion thereof, adjacent crotch edge 22. The absorbent garment of the invention also preferably is provided with waist elastic material 32 optionally in the first and second waist regions, 12, 14, respectively, to enable and assist in stretching around the wearer. The waist elastics 32 may be similar structures or different to impart similar or different elastic characteristics to the first and second waist regions 12, 14 of the garment. In general, the waist elastics may preferably comprise foam strips positioned at the first and second waist regions 12, 14, respectively. Such foam strips preferably are about ½ to about 1½ inches along the dimension parallel to longitudinal axis 100 and about 3-6 inches along the dimension parallel to lateral axis 102. The foam strips preferably are positioned between the top sheet 24 and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. Preferably at least a portion of foam strips 32 are stretched 50-150%, preferably 100% more than their unstretched dimension before being adhesive-lessly secured between the back sheet 26 and top sheet 24. U.S. Pat. No. 4,515,595 to Kievit et al. and U.S. Pat. No. 4,816,025 to Foreman illustrate other embodiments of elasticized waist features of absorbent garments, and are hereby incorporated by reference in their entirety.

Each edge 22 that forms the leg openings preferably is provided with an adjacent leg elastic containment system 30. In an exemplary embodiment, three strands of elastic threads are positioned to extend adjacent to leg openings between the top sheet 24 and the back sheet 26. Any suitable elastomeric material exhibiting at least an elongation (defined herein as (L_{S}−L_{R})/L_{R} where L_{S} is the stretch length of an elastic element and L_{R} is retracted length, multiplied by 100 to obtain percent elongation) in the range of 5%-35%, preferably in the range of 200%-400%, can be employed for the leg elastics 30. The leg elastics 30 may be attached to the absorbent article 10 in any of several ways, which are known in the art. For example, the leg elastics 30 may be ultrasonically bonded, heat bonded or pressure sealed using a variety of bonding patterns. Various commercially available materials can be used for the elastics, such as natural rubber, butyl rubber or other synthetic rubber, urethane, elastomeric materials such as LYCRA (DuPont), GLOSPAN (RadiciSpandex) or SYSTEM 7000 (Fulflex). Alternatively elastics may be provided as a preformed laminates and the laminate attached to the garment by an adhesive-less bond.

Additional elastics (not shown) may also be incorporated into the chassis layer, top sheet 24 or back sheet 26 adjacent the leg holes to form conventional (i.e., non-standing) leg gathers, as is known in the art. Conventional gathers contract the garment 10 around the wearer's legs and body to prevent leakage. U.S. Pat. Nos. 3,860,003 and 4,081,301 issued to Buell, U.S. Pat. No. 4,695,278 issued to Lawson, U.S. Pat. No. 4,808,177 issued to Des Marais, U.S. Pat. No. 4,795,454 issued to Dragoo, and U.S. Pat. No. 4,938,755 issued to Foreman illustrate other embodiments of leg cuffs and gathers in absorbent garments, and the disclosures of each of these patents are hereby incorporated by reference in their entirety.

It is often desirable for an absorbent garment to contract around various parts of the wearer's body to provide improved comfort and exudate containment. In addition to the leg gathers 30 and waist elastic 32, tummy elastics (not shown) may be incorporated into the garment 10 to contract the garment 10 about the wearer's waist and stomach. Such elastics are typically stretched as they are joined to the garment 10 so that the contraction of the elastics causes the garment 10 to contract about the wearer. The elastics may also be applied in an unstretched state then mechanically stretched to create an elasticized region (often called a zero-strain laminate). The elastics may also be applied in an inelastic state then heat activated to cause them to become elasticized. The tummy elastics may be made from material as synthetic rubber, elastomers, LYCRA® elastomers (available from E. I. DuPont du Nemours and Company, a business having offices in Wilmington, Del.), polyurethane, heat shrinkable polymer ribbons or any other suitable elastic material or composite.

Elastic materials may be attached to the absorbent garment by ultrasonic or thermal bonding as discussed above. Alternatively the elastic may comprise a polymeric material having both elastic and adhesive properties that self-adheres to a substrate with, for example, the application of pressure. Such polymeric materials having both elastic properties and the ability to self adhere to a substrate are described in U.S. Pat. No. 4,761,198 to Salerno and U.S. Pat. No. 4,259,220 to Bunnelle et al., the disclosures of which are incorporated herein by reference in their entirety. In some embodiments a polymer having elastic properties may be extruded directly onto the substrate and attach to the substrate as it cures.

In another embodiment the elastic materials may be provided with a coating which adheres to a surface upon the application of the pressure as described in U.S. Pat. No. 5,503,908 to Faas the disclosure of which is incorporated herein by reference in its entirety. Alternatively, the elastic material may be provided with a coating that may be activated by heat or UV light, for example, and caused to adhere to a substrate. U.S. Pat. No. 4,081,301 to Buell discloses such an elastic having a heat activated coating that may be adhered to a substrate upon the application of heat. The Buell patent is incorporated herein in its entirety.

Alternatively, the elastic material may be provided as a laminate material. The laminate may be prepared by disposing one or more elastic strands between a first substrate and a second substrate. In one embodiment the elastic strand may be held in position by application of an adhesive in a laminate formation process and supplied to the absorbent garment assembly area as a preformed laminate. One or more preformed laminates may then be attached to the absorbent garment by one or more adhesive-less bonds. It is preferable to eliminate the use of an adhesive (e.g. a discrete intermediary substance that joins at least a portion of the surface of two or more components) and directly join integral portions of the components. However, the use of a preformed laminate, constructed using adhesive at an offline laminate assembly station and supplied to garment assembly process as a preformed laminate for attachment to the garment by one or more adhesive-less bonds does permit elimination of adhesive application in an absorbent garment assembly production line.

The fastening elements, preferably a fastening system 34 (e.g., tab 34) of the preferred embodiment, is attached to the first waist region 12. It preferably comprises a tape tab or mechanical fasteners 36. However, any fastening mechanism known in the art will be acceptable. A landing zone or attachment area 38 preferably is provided in the second waist region 14 to permit attachment of the first waist region 12 to the second waist region 14. Preferably the fastening mechanism permits opening and closing so that the diaper may be checked for soiling without compromising the ability to reuse the fastener. In some embodiments the area adjacent the fastener may be reinforced to facilitate opening and closing the fasteners. Alternatively, other absorbent article fastening systems are also possible, including tapes, adhesives, safety pins, buttons, and snaps.

In some embodiments the tabs 34 may be formed as part of the front sheet 24, back sheet 26 or both with tape areas or mechanical fasteners 36 attached to the tabs 34 by adhesive-less bonds. In other embodiments one or both of the tabs 34 and landing zone or area of attachment 38 may be formed as separate components and attached to the absorbent garment by one or more adhesive-less bonds such as ultrasonic bonds or thermal bonds or a combination thereof.

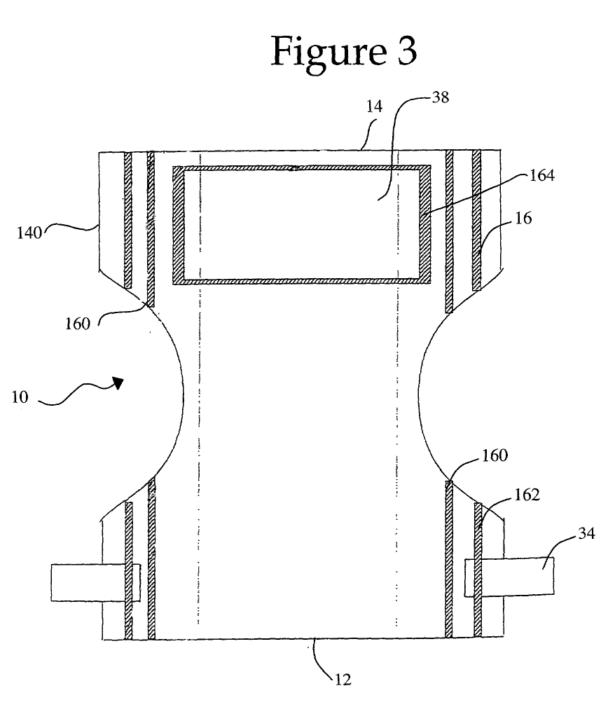

In the embodiment of the invention shown in FIG. 3, the separate component tabs 34 are attached to the absorbent garment by ultrasonic weld seams 162. The tabs 34 may be disposed adjacent the top sheet 24, adjacent the back sheet 26 or there between. The ultrasonic weld seam 162 may directly join two or more component layers at the weld site. Thus, for example, the top sheet 24 back sheet 26 and tab 34 may be directly joined to each other in a single bonding event. Likewise, the landing zone or area of attachment 38 may be joined to the back sheet 24 or to the back sheet 24 and underlying layers by a ultrasonic weld 164. The layers assembled together when the bonding event occurs and the energy imparted are some factors that determine the number of layers bonded together. In another embodiment, thermal bonding may be used. The positioning and length of bonds 162 and 164 are exemplary. For example, bond 164 may alternatively be placed inward from the edges of landing zone 38, to follow a portion of the perimeter, have a curved shape or be multiple bonds, for example. As discussed above, the pattern of the bond and the dimensions of the bonded area may be precisely controlled to optimize bond strength, minimizing stiffening and improve aesthetics by adding decorative effects and in some embodiments provide re-enforcement.

FIG. 3 also shows ultrasonic weld seams 160. Ultrasonic weld seams 160 secure the top sheet 24 and the back sheet 26 in position with respect to each other and may be an alternative to the pin bonding 130 shown in FIG. 2. In some embodiments the ultrasonic weld seams 160 may also serve a re-enforcement function.

As stated previously, the invention has been described in connection with a diaper. The invention, however, is not intended to be limited to application only in diapers. Specifically, the absorbent cores of the preferred embodiments may be readily adapted for use in other absorbent garments besides diapers, including, but not limited to, training pants, feminine hygiene products and adult incontinence products.

The underlying structure beneath the top sheet 24 may include, depending on the diaper construction, various combinations of elements, but in each embodiment, it is contemplated that the absorbent garment will preferably include an absorbent core 28. For example, additional layers may be disposed between the top sheet 24 and absorbent core 28, or between absorbent core 28 and back sheet 26 or a combination thereof. The additional layer(s) may include a fluid transfer layer, a fluid handling layer, a storage layer, a wicking layer, a fluid distribution layer, and any other layer(s) known to those having ordinary skill in the art. These layers may be fixed into position with respect to each other by adhesive-less bonding of two or more layers. In embodiments in which multiple layers are bonded, the layers may be assembled forming a stack of components and bonded by an adhesive-less bond in a single bonding event. Alternatively, selected layers may be joined at predetermined positions by adhesive-less bonds and the so joined layers further assembled with additional layers which are then fixed into position with respect to each other by adhesive-less bonds.

Although the absorbent core 28 depicted in FIG. 1 has a substantially rectangular cross-sectional and plan view shape, other shapes may be used, such as a “T” shape or an hourglass shape. The shape of the absorbent core 28 may be selected to provide the greatest absorbency with a reduced amount of material. The absorbent core may be associated with the top sheet 24, back sheet 26, or any other suitable part of the garment 10.

As shown in FIG. 2, in one embodiment the absorbent core 28 may be held in position by an adhesive-less bond 150 between the top sheet 24 and back sheet 26 adjacent the perimeter 125 of the absorbent core 28. As shown in FIG. 2 an ultrasonic weld bond 150 between the top sheet 24 and back sheet 26 surrounds the entire perimeter of the absorbent core 28 in effect forming a pocket substantially the same size as the absorbent core 28 holding the absorbent core in position with respect to the top sheet 24 and back sheet 26. Alternatively, the absorbent core 28 may be attached to the top sheet 24, back sheet 28 or both by one or more adhesive-less bonds. Such bonding may be selectively positioned to minimize the surface area of the absorbent core 28 engaged in bonding.

The entire absorbent core 28 may be enclosed within a tissue wrapping, as disclosed in U.S. Pat. No. 6,068,620, the disclosure of which is incorporated by reference herein in its entirety. In some embodiments the tissue wrapping may be attached to one or more other components of the garment 10 by an adhesive-less bond. Alternatively, the absorbent core 28 may be “sandwiched” between two layers of material, the assembly positioned adjacent one or more other layers of the garment such as the top sheet 24, and/or back sheet 28, for example, and the multiple layers bonded in a single bonding event. The two layers of material adjacent the core may serve principally to facilitate positioning of the absorbent core 28 or may include a layer that facilitates transfer of liquid to the core, for example. Skilled artisans are capable of designing and wrapping a suitable absorbent core 28 of the invention, using the guidelines provided herein. In some embodiments the wrapping may be attached to another component such as the top sheet 24 or back sheet 26 by an adhesive-less bond.

Any suitable absorbent material may be used for absorbent core 28. Absorbent cores containing a mixture of fibrous material and superabsorbent polymers (SAP) are well known in the art and described, for example, in U.S. Pat. Nos. 5,281,207, and 6,068,620 to Chmielewski, and U.S. Pat. No. 5,863,288, to Baker, the disclosures of each of which are herein incorporated by reference in their entirety and in a manner consistent with this disclosure. The fibrous material can be any fibrous material capable of absorbing fluids, and capable of retaining SAP particles within its matrix. Preferred fibrous materials may be selected from tow fibers, cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof. In addition, rayon, Courtauld's LYOCELL, polyacrylonitrile, cotton fibers and cotton linters are alternatively preferred. The remaining fibers, surface-modified polyolefin/polyester bicomponent fibers, and surface-modified polyester/polyester bicomponent fibers are also believed to be effective fibrous materials for use in the invention.

Any superabsorbent polymer (SAP) now known or later discovered may be used in absorbent core 28, so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

The components described herein are assembled to form an absorbent garment. It has heretofore been known to conventionally use adhesives to hold the components in the desired positions. Adhesives are defined herein as discrete compositions that join at least a portion of the surfaces of two or more components in a substantially fixed position with respect to each other. In preferred embodiments of the present invention, however, no adhesive is used in assembling the absorbent garments of the invention. In other embodiments a preformed laminate(s) that, as supplied, has been formed using adhesives may be used for some components such as the leg gathers, but no adhesive is used during the assembly of the laminate component with other components to form the garment. In another embodiment components having an integral portion that has adhesive properties may be used to attach the component to one or more other components, but no adhesive is applied in the component assembly process.

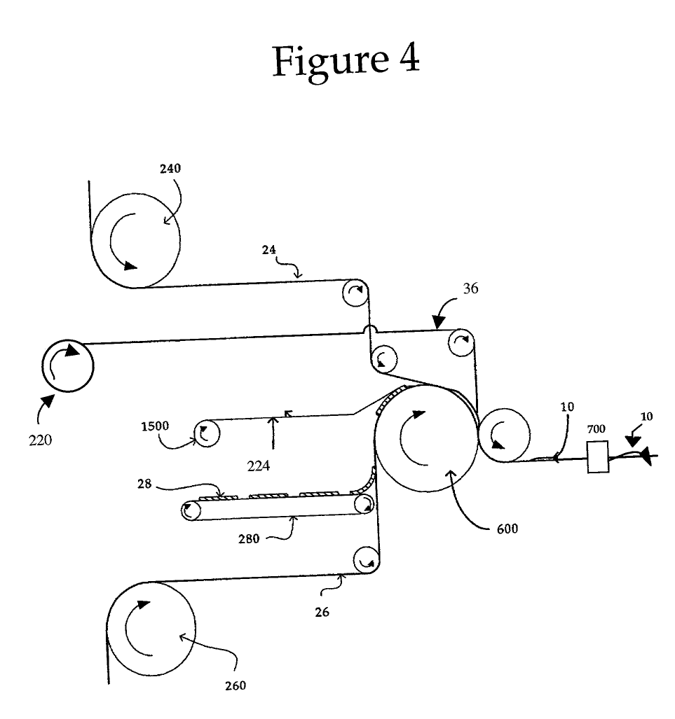

FIG. 4 is a schematic of a preferred apparatus for assembling an absorbent garment in accordance with the invention. In this embodiment the top sheet material 24 and the back sheet material 26 and the absorbent core 28 are brought into contact with each other at forming station 600. A continuous supply of back sheet material 26 is provided along the machine direction to the forming station 600 via the back sheet supply roller to 260. Absorbent cores 28 preferably are supplied via absorbent core supply mechanisms to 280 and deposited on the back sheet material 26 (or other layer or layers inter posed there between) on the surface that will eventually become the inner surface of the back sheet 26 of the garment 10. In the embodiment shown, the absorbent core 28 is positioned on the back sheet material 26, but is not bonded at this point. Alternatively, a bonding station may be provided for affixing the absorbent core 28 to the back sheet 26 using an adhesive-less bond prior to positioning of the top sheet.

A continuous supply of top sheet material 24 is provided substantially in the machine direction to forming station 600 via top sheet supply roller 240. Top sheet supply material 24 preferably overlays the back sheet material and encases the absorbent core. The top sheet 24 may be bonded to the back sheet 26 in an adhesive-less bond around the perimeter 125 of the absorbent core 28 at this point or otherwise be operatively associated with the back sheet material 26, absorbent core material 28 or both. Alternatively, these materials may be positioned with respect to each other and bonded at a later location. In some embodiments a fastener supply mechanism 220 may supply fasteners 36 to the supply station 600, an elastics supply mechanism 1500 may supply one or more elastic strands 224 or laminates to the forming station 600 or both.

In one embodiment the top sheet 24, the back sheet 26, the absorbent core 28, fasteners 36 and elastic components are fed into the forming station 600 such that the various components are positioned in the desired location with respect to the other components to form an absorbent garment as described herein. In the embodiment shown in FIG. 4, the components so assembled are passed to bonding station 700 where one or more adhesive-less bonds are executed to secure the components in the desired positions with respect to each other. In preferred embodiments the bonding station will execute ultrasonic bonding, thermal bonding, pressure bonding or a combination thereof.

As shown in FIG. 4 bonding may be accomplished in a single station in one embodiment. In other embodiments it may be desirable to use multiple bonding stations. For example, the back sheet material 26 may be provided in the machine direction to the forming station 600 and absorbent core 28 supplied to the forming station by absorbent core supply mechanisms 280 and deposited on the back sheet on the surface that will eventually become the inner surface of the back sheet. The top sheet material 24 may be supplied to the forming station in such a manner that it overlays the back sheet material 26 and encases the absorbent core 28. The top sheet material may then be bonded to the back sheet material around the perimeter 125 of the absorbent core 28 (FIG. 2) to create a pocket securing the position of the absorbent core with 28 with respect to the top sheet 24 and the back sheet 26 to form an absorbent core assembly. The elastic components and fasteners 36 may be assembled with the absorbent core assembly and attached at separate forming station(s) (not shown). Further assembling and bonding steps of the elastic component(s) and fasteners 36 may occur at separate stations, occur at a single station or at multiple positions within a single station, for example.

In embodiments employing multiple adhesive-less bonding steps, tabs may be attached to or integrally formed with the top sheet material 24 and/or back sheet material 26 at the left and right rear side edges 18 (FIG. 1). The tabs support fastening elements 36. Any fastening elements may be used, including inter alia, hooks, loops, tapes, clips, snaps, and the like. As mentioned above, the fastening elements, tabs (if separate tabs are used) or both may be attached at a tab bonding station by any of the adhesive-less bonding methods discussed herein. The tab bonding station may be a stand alone station or a portion of another bonding station.

At least one target device 38 can be disposed at or near the front waist region 14 of the garment 10 whereby the at least one target device forms a landing zone and otherwise cooperates or associates with the fastening elements. Suitable target devices include, hooks, loops, tape, landing zones, clips, snaps and the like. Those skilled in the art are capable of determining the position of the tabs and landing zone devices for associating the front waist region 14 with the rear waist region 12 to form a parts type absorbent garment 10, using the guidelines provided herein. The target device may be positioned and bonded to the absorbent garment by an adhesive-less bond at the tab bonding station or at a target device bonding station.

The elastics may be attached using adhesive-less bonds as discussed above, attached by the application of pressure to those elastic materials having a pressure sensitive integral portions of the elastic, or attached by activation of an integral part of the elastic by heat or UV light, for example at a separate bonding station or a portion of another bonding station.

As discussed above, other layers and liners may be included in addition to the top sheet 24, the back sheet 26, and the absorbent core 28. Likewise, these additional layers may be positioned by being fed into forming station 600 and the multiple layers of the assembled garment bonded by the formation of one or more adhesive-less bonds in bonding station 700. Alternatively, various layers may be assembled and bonded prior to combination with other components of the absorbent garment. In such embodiments multiple adhesive-less bonding stations may be employed. Sequential bonding stations may offer some advantages in compatibility with known commercial assembly equipment and practice.

After bringing the components together at forming station 600, and executing one or more adhesive-less bonds at bonding station 700, or alternatively, after the top sheet 24, back sheet 26, absorbent core 28, any other layers adjacent these components, fasteners, and elastics have been assembled and bonded, the absorbent garment preferably is further processed by cutting and folding, and/or carrying out other processing procedures (e.g. providing standing leg gathers 40 etc.). For example the top sheet material 24 and the back sheet material 26 preferably are cut to form leg hole cutouts on each side of the assembly. Any suitable cutting device may be used such as such as fixed blades, cutting drums or reciprocating cutters. Further in some embodiments a single device may provide blades to cut leg hole cut outs and also provide anvil(s) to form ultrasonic bonds or heated element(s) to form thermal bonds, or activation source(s) for bonding components having composition displaying adhesive properties upon activation, and thus serve as both a bonding station and a cutting station. Further additional adhesive-less bonding may be desirable to provide seals along the respective front rear left and right side arcuate edges of garment 10. Pressure devices also may be employed to provide suitable seals when components have integral pressure sensitive adhesive portions. Alternatively, heat or UV light sources may be employed to activate adhesion of components with integral parts amenable to attachment upon such activation.

Additional processing steps may also take place to fasten the front and rear waist edges 12, 14 together. For example, garment 10 may be folded in half and then adhesive-less bonding used to join the respective front and rear side edges 18 to one another thereby forming disposable pants type garment 10. Once the garment 10 is assembled, final processing steps may include folding and packaging steps.

One skilled in the art will understand that the locations of the various parts of the invention during the assembly process and intervals at which the parts are placed on the assembly are selected such that the various parts are in the proper location in the final product. In addition other parts such as waist elastic element 32 or standing leg gathers 40 may be incorporated into the assembly during the assembly process, and other processes, such as folding and packaging may be incorporated in the assembly process. Also, it should be understood that that any suitable method may be used to introduce the various components of the garment 10 of the assembly line, such as rollers, vacuum drums, conveyors or reciprocating stamps. Finally it should also be understood that the order of the various forming procedures may be modified, combined or rearranged to provide for various assembly sequences that will provide substantially the same finished product, and that the variations are within the scope and spirit of the present invention and are within the knowledge and skill of those skilled in the art in the light of the present teaching. For example, as discussed above multiple layers of material may be bonded together in a single adhesive-less bonding event or one or more layers may be bonded together and subsequently bonded to one or more other layers. Other modifications will be apparent to those skilled in the art.

The invention has been described in connection with the preferred embodiments, these embodiments, however, are merely for example and the invention is not restricted thereto. It will be understood by those skilled in the art that other variations and modifications can easily be made within the scope of the invention as defined by the appended claims.

What is claimed is:

1. An absorbent garment having a longitudinal and a lateral dimension comprising:
a top sheet;
a back sheet whereby the top sheet and the back sheet form a first waist region, a second waist region longitudinally opposite the first waist region and a crotch there between; and
an absorbent core disposed between the top sheet and back sheet
whereby the top sheet, the back sheet and
the absorbent core are secured to one another by at least one adhesive-less bond.

2. The absorbent garment of claim 1, wherein a portion of the at least one adhesive-less bond is an ultrasonic bond.

3. The absorbent garment of claim 1, wherein a portion of the at least one adhesive-less bond is a thermal bond.

4. The absorbent garment of claim 1, wherein the absorbent core has a perimeter edge and the adhesive-less bond is between the top sheet and the back sheet adjacent a portion of the perimeter edge thereby securing the absorbent core with respect to the top sheet and the back sheet.

5. The absorbent garment of claim 1, wherein the back sheet is a film comprising at least one material selected from the group consisting of polyethylene, polypropylene, polyester, nylon, polyvinyl chloride and mixtures thereof.

6. The absorbent garment of claim 1, wherein the top sheet is a non-woven spunbond of at least one material selected from the group consisting of polypropylene, polyethylene, nylon, polyester and mixtures thereof.

7. The absorbent garment of claim 1, wherein the top sheet is a carded web of at least one material selected from the group consisting of polypropylene, polyethylene, nylon, polyester and mixtures thereof.

8. The absorbent garment of claim 1, further comprising at least one layer, the layer being at least one layer selected from the group consisting of an outer top sheet panel, chassis, back sheet panel, liner, transfer layer, laminate, graphics panel, and combinations thereof, whereby the at least one layer is attached to the absorbent garment by at least one adhesive-less bond.

9. The absorbent garment of claim 1 further comprising a pair of tabs extending laterally away from an opposite lateral side of the first waist region, at least a portion of each tab including a fastening element, and one or more target devices attached to the garment in the second waist region, wherein the fastening elements and the target devices are capable of attaching to one another, the one or more target devices being located so that the first and second waist region of the garment may be joined to secure the garment on the intended wearer; whereby the fastening elements are attached to the tabs, and the one or more target devices are attached to the absorbent garment by adhesive-less bonds.

10. The absorbent garment of claim 9, wherein the tabs are individual components attached to the absorbent garment by adhesive-less bonds.

11. The absorbent garment of claim 1, further comprising at least one elastic strand.

12. The absorbent garment of claim 11, wherein at least one elastic strand is attached to the garment by an adhesive-less bond.

13. The absorbent garment of claim 11, wherein the elastic strand is precoated with a polymer, the polymer upon activation forming a bond between the elastic strand and the absorbent garment.

14. The absorbent garment of claim 11, wherein the elastic strand is extruded onto a portion of the absorbent garment and adhered to the absorbent garment.

15. The absorbent garment of claim 11, wherein the elastic strand is disposed in a laminate comprising a first substrate and a second substrate with the elastic strand disposed between the first and second substrates and wherein the laminate is attached to the absorbent garment by an adhesive-less bond.

16. An apparatus for making a disposable absorbent garment comprising:
a top sheet supply mechanism for supplying a top sheet;
a back sheet supply mechanism for supplying a back sheet;
an absorbent core supply mechanism for supplying an absorbent core;
a forming station which receives the top sheet from the top sheet supply mechanism, the back sheet from the back sheet supply mechanism and the absorbent core from the absorbent core supply mechanism and disposes the absorbent core between the top sheet and the back sheet; and
at least one adhesive-less bonding station that creates at least one adhesive-less bond to secure the top sheet, the back sheet and the absorbent core in a position with respect to each other.

17. The apparatus of claim 16, wherein a portion of the at least one adhesive-less bond is an ultrasonic bond and the at least one adhesive-less bonding station comprises at least one ultrasonic bonding device.

18. The apparatus of claim 16, wherein a portion of the at least one adhesive-less bond is a thermal bond and the at least one adhesive-less bonding station comprises at least one thermal bonding device.

19. The apparatus of claim 16 further comprising:
a fastener system supply mechanism for supplying a fastener system; and
a fastener system attachment station for attaching the fastener system to the absorbent garment with at least one adhesive-less bond.

20. The apparatus of claim 19, wherein the fastener system supply mechanism supplies at least one fastening element and at least one target device; and the fastener system attachment station receives the at least one fastening element and the at least target device and attaches the at least one fastening element to a tab that is an extension portion of at least one of the top sheets and back sheets, and attaches the at least one target device to a second portion of at least one of the top sheets and back sheets.

21. The apparatus of claim 19, wherein the fastener system attachment station has a fastener element attachment portion and a target device attachment portion.

22. The apparatus of claim 19, wherein the fastener system supply mechanism supplies at least one fastening element and at least one target device, and at least one of the at least one fastening element and the at least one target device is attached to at least one separate component tab by an adhesive-less bond, and wherein the fastener system attachment station attaches the at least one separate component tab to the absorbent garment by an adhesive-less bond.

23. The apparatus of claim 16, further comprising:
an elastic strand supply mechanism; and
an elastic strand attachment station,
wherein the elastic strand supply mechanism supplies at least one elastic strand and the elastic strand attachment station receives at least one elastic strand and attaches it to the absorbent garment by an adhesive-less bond.

24. The apparatus of claim 16, further comprising:
an elastic strand supply mechanism; and
an elastic strand attachment station,
wherein the elastic strand supply mechanism supplies an elastic strand precoated with a polymer, the polymer upon activation forming a bond between the elastic strand and the absorbent garment.

25. The apparatus of claim 16 further comprising:
an elastic strand supply mechanism; and
an elastic strand attachment station,
wherein the elastic strand is extruded from the elastic strand supply mechanism onto a portion of the absorbent garment and adhered to the absorbent garment at a portion of the elastic strand attachment station

26. The apparatus of claim 23, wherein the elastic strand supply mechanism supplies at least one elastic strand disposed in an elastic strand laminate comprising a first substrate, and a second substrate with the elastic strand disposed between the first and second substrate to the elastic strand attachment station, and wherein the elastic strand attachment station attaches the laminate to the absorbent garment with at least one adhesive-less bond.

27. The apparatus of claim 26, wherein the elastic strand attachment station attaches a plurality of elastic strand laminates to the absorbent garment by at least one adhesive-less bond.

28. A method for manufacturing an absorbent garment comprising:
providing a top sheet, a back sheet and an absorbent core;
disposing the absorbent core between the top sheet and the back sheet; and
fixing the top sheet, back sheet and absorbent core in a position with respect to each other with at least one adhesive-less bond.

29. The method of claim 28, wherein a portion of the at least one adhesive-less bond is an ultrasonic bond.

30. The method of claim 28, wherein a portion of the at least one adhesive-less bond is a thermal bond.

31. The method of claim 28, further comprising: attaching a layer selected form the group consisting of at least one of an outer top sheet panel, chassis, back sheet panel, liner, transfer layer, laminate and graphics panel, to the absorbent garment by at least one adhesive-less bond.

32. The method of claim 28, comprising attaching a fastener system to the absorbent garment with at least one adhesive-less bond.

33. The method of claim 32, wherein the fastener system includes at least one fastening element and at least one target device, and wherein at least one of the fastening element or target device is attached to a tab that is an extension portion of at least one of the top sheet and back sheet.

34. The method of claim 32, wherein the fastener system includes at least one fastening element and at least one target device and at least one of the fastening element and target device is attached to at least one separate component tab and the at least one separate component tab is attached to the absorbent garment by an adhesive-less bond.

35. The method of claim 28 further comprising attaching at least one elastic strand to the absorbent garment by an adhesive-less bond.

36. The method of claim 35, wherein the at least one elastic strand is extruded onto a portion of the absorbent garment.

37. The method of claim 35, wherein the elastic strand is precoated with a polymer, the polymer upon activation forming a bond between the elastic strand and the absorbent garment.

38. The method of claim 28, wherein the at least one strand is disposed in a laminate comprising a first substrate and a second substrate with the elastic strand disposed between the first and second substrates, and wherein the laminate is attached to the absorbent garment by an adhesive-less bond.

39. The method of claim 38, wherein a plurality of laminates are attached to the absorbent garment by at least one adhesive-less bond.

40. A core assembly for an absorbent garment comprising:
a top sheet;
a back sheet; and
an absorbent core,
wherein the absorbent core has a perimeter edge and is disposed between the top sheet and back sheet, and the top sheet and back sheet are bonded by an adhesive-less bond adjacent a portion of the perimeter edge, thereby substantially holding the absorbent core in a relatively fixed position with respect to the top sheet and the back sheet.

41. The core assembly of claim 40, wherein a portion of the at least one adhesive-less bond is an ultrasonic bond.

42. The core assembly of claim 40, wherein a portion of the at least one adhesive-less bond is a thermal bond.
